(19) United States
(12) Patent Application Publication
(10) Pub. No.: US 2008/0114436 A1
(43) Pub. Date: May 15, 2008

(54) ANEURYSM COVERING DEVICES AND DELIVERY DEVICES

(76) Inventors: Martin S. DIECK, Cupertino, CA (US); Brian B. Martin, Felton, CA (US)

Correspondence Address: LEVINE BAGADE HAN LLP, 2483 EAST BAYSHORE ROAD, SUITE 100, PALO ALTO, CA, 94303, US

(21) Appl. No.: 11/840,679

(22) Filed: Aug. 17, 2007

Related U.S. Application Data
(60) Provisional application No. 60/822,741, filed on Aug. 17, 2006.

(51) Int. Cl. A61F 2/06

(52) U.S. Cl. 623/1.11; 623/1.13; 623/1.15

(57) ABSTRACT

Devices are provided for isolating an aneurysm from the blood vessel, particularly berry aneurysms within the cerebral vasculature. Embodiments of such devices have improved manufacturability, deliverability and isolation of the aneurysm. Delivery systems are also provided for such devices and other devices which may benefit from orientation adjustment during delivery.

### CROSS-REFERENCE TO RELATED APPLICATIONS

This application is a non-provisional of U.S. Provisional Application No. 60/822,741 filed on Aug. 17, 2006 entitled ANEURYSM COVERING DEVICES AND DELIVERY DEVICES. The entirety of which is hereby incorporated reference.

### BACKGROUND OF THE INVENTION

The term aneurysm refers to any localized widening or outpouching of an artery, a vein, or the heart. All aneurysms are potentially dangerous since the wall of the dilated portion of the involved vessel can become weakened and may possibly rupture.

A common type of aneurysm is a brain aneurysm. Brain aneurysms are widened areas of arteries or veins within the brain itself. These may be caused by head injury, an inherited (congenital) malformation of the vessels, high blood pressure, or atherosclerosis. A common type of brain aneurysm is known as a berry aneurysm. Berry aneurysms are small, berry-shaped outpouchings of the main arteries that supply the brain and are particularly dangerous since they are susceptible to rupture, leading to often fatal bleeding within the brain. Brain aneurysms can occur at any age but are more common in adults than in children.

A variety of devices have been developed to cover such aneurysms, including stentlike devices having a one-sided covering or patch to cover the opening of the aneurysm along the blood vessel. However, such devices are often difficult to construct and deploy. In particular, these one-sided coverings need to be correctly oriented and deployed so as to cover the aneurysm opening. This is challenging in that the vascular anatomy preceding most aneurysms is very tortuous and long and therefore difficult to control and transmit torque for precise delivery. Therefore, improved devices for treatment of aneurysms are desired along with improved delivery devices and methods. At least some of these objectives will be met by the present invention.

### SUMMARY OF THE INVENTION

The description, objects and advantages of the present invention will become apparent from the detailed description to follow, together with the accompanying drawings.

### BRIEF DESCRIPTION OF THE DRAWINGS

FIGS. 1A-1E illustrate an embodiment of a covering device along with an example of its manufacture.

FIG. 2A illustrates a blood vessel having a berry aneurysm.

FIG. 2B illustrates a covering positioned with the blood vessel of FIG. 2A so that the covering covers the opening of the aneurysm.

FIGS. 2C-2D illustrates a delivery system having a fill tube coupled with the covering device.

FIG. 3 illustrates another embodiment of a covering device of the present invention.

FIGS. 4A-4B illustrate a covering device having an oval shaped covering.

FIGS. 5-7 illustrate alternative embodiments having coverings of different shapes and sizes.

FIG. 8 illustrates an embodiment of a covering having a diamond shape with joints.

FIG. 9 illustrates an embodiment of a covering device having a covering attached to two rings, each by a coaxial strut.

FIGS. 10A-10B illustrates an embodiment of a covering device having a covering attached to each ring by a curved strut.

FIGS. 11A-11D illustrates an embodiment of a covering device including two rings having a C-shape, wherein each ring is connected to two struts which are coupled to the covering at a common location.

FIG. 12 illustrates some example features which may be included in a covering.

FIG. 13 illustrates a variety of methods for providing radiopacity

FIG. 14 illustrates an example of device material having radiopaque material incorporated therein.

FIG. 15 illustrates an embodiment of a covering device constructed from mesh or a cut tube stent and a covering formed from a flexible material.

FIG. 16 illustrates a method of joining two materials.

FIG. 17 illustrates a covering positioned within a bifurcated blood vessel so that the covering covers the aneurysm.

FIGS. 18A-18B illustrates a push-style delivery system.

FIG. 19 illustrates a pull-style delivery system.

FIGS. 20A-20C illustrates a sheath-style delivery system.

FIG. 21 illustrates the positioning of a guidewire within an aneurysm in a blood vessel.

FIG. 22 illustrates a sheath-style delivery system having a force conversion mechanism.

FIGS. 23A-23B illustrates an embodiment of a force conversion mechanism.

FIG. 24 illustrates another embodiment of a force conversion mechanism.

FIGS. 25A-25D illustrate an embodiment of a delivery system which provides for rotation of the covering device with the use of a pulley.

FIG. 26 illustrates another embodiment of a pulley.

FIGS. 27A-27C illustrate an embodiment of a delivery system which provides for rotation of the covering device with the use of a threaded portion.

### DETAILED DESCRIPTION OF THE INVENTION

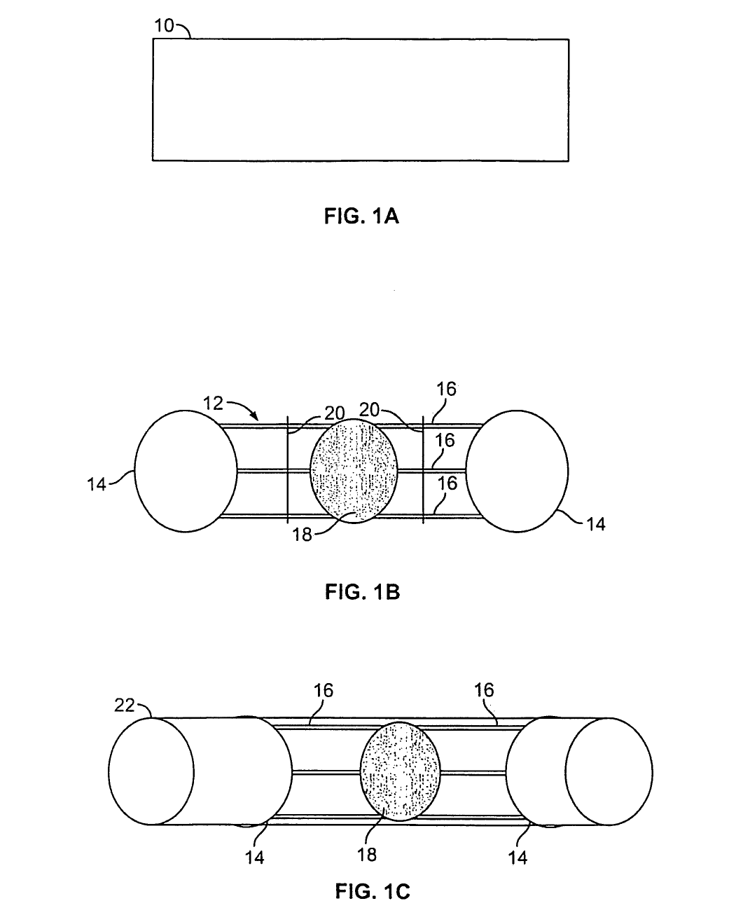

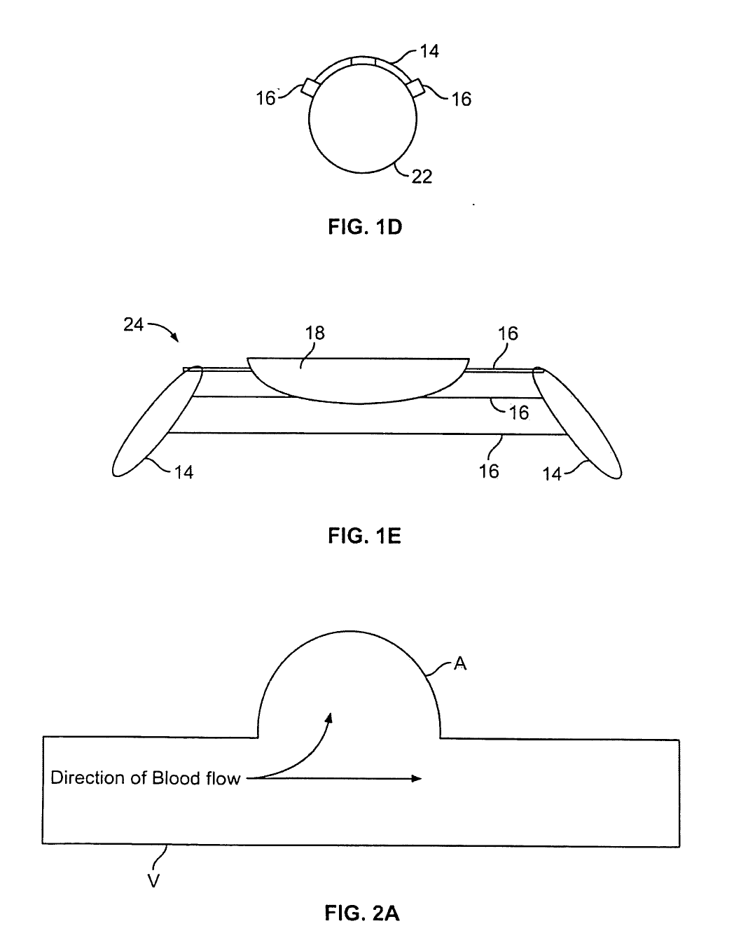

A variety of covering devices are provided which may be used to treat an aneurysm, particularly a berry aneurysm. One embodiment of such a covering device, along with an example of its manufacture, is illustrated in FIGS. 1A-1E. In this embodiment, the device is laser cut from a sheet 10 of material. Example materials include nitinol, stainless steel, cobalt, chromium, and tantalum, to name a few. FIG. 1A illustrates such a sheet 10 of material. FIG. 1B illustrates an example cutout 12 from the sheet 10. In this embodiment, the cutout 12 includes rings 14, struts 16 and a covering 18. The rings 14 and/or struts 16 may vary in length to allow for improved delivery and final stability. The covering 18 may be solid or split to facilitate folding and delivery. In other embodiments, the covering 18 comprises a scaffold, such as a ring or hoop, which is covered by ePTFE or an elastic material or metal or the covering 18 is not cut from the sheet 10 and is added in a later step of the manufacture. Further, optional bands 20 may be added to encourage expansion in a curved direction. The bands 20 may be made of plastic and may be flexible but become tight against the force of expansion contributing to the expansion in a curved direction. Also, struts 16 may optionally be coated with a polymer to reduce friction during delivery. 25

Referring to FIG. 1C, the cutout 12 is then mounted on a shaping device 22, such as a cylinder or mandrel. FIG. 1D illustrates a cross-section of FIG. 1C. The cutout 12 is then set in this shape, such as by the application of heat and by bending over the shaping device 22, to form the covering device 24. FIG. 1E illustrates the covering device 24 removed from the shaping device 22.

Such construction of the device 24 from a flat sheet 10 provides significant advances in manufacturability. This is due to the ease in which a machining process can be conducted in two dimensions compared to three dimensions. However, it may be appreciated that the covering device 24 may alternatively be constructed from a tube or from shaped wire, for example.

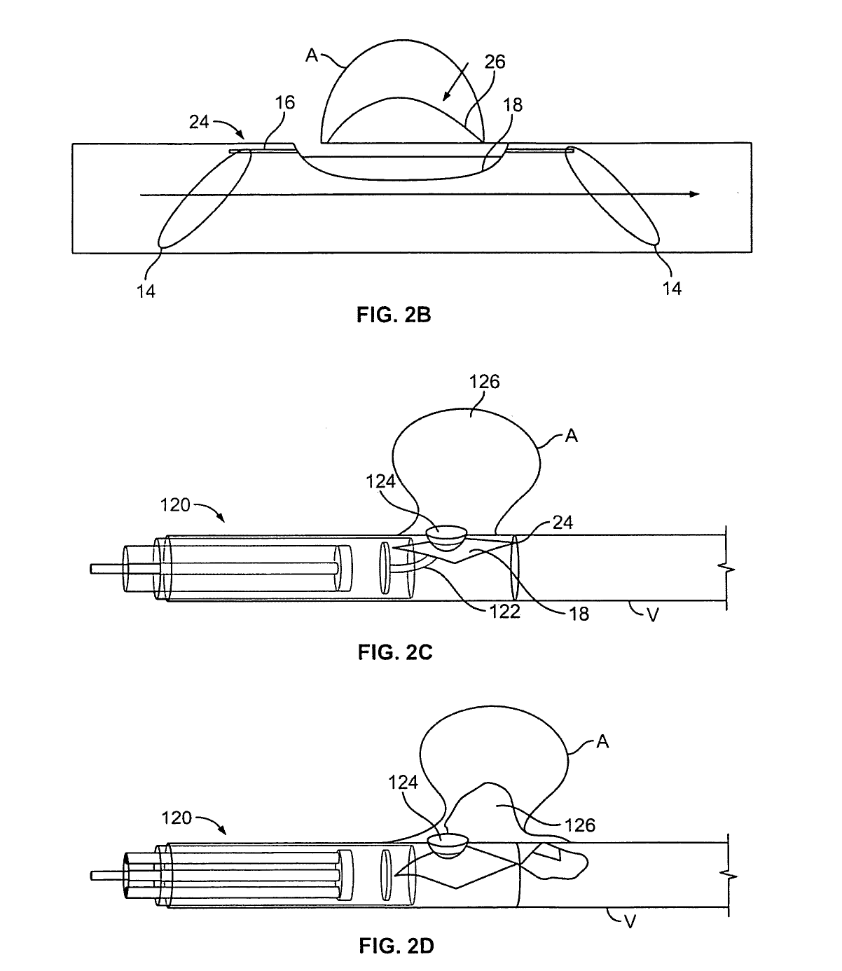

FIG. 2A illustrates a blood vessel V having a berry aneurysm A. Blood is shown flowing through the vessel V and into the aneurysm A. FIG. 2B illustrates the covering device 24 of FIG. 1E positioned within the blood vessel V so that the covering 18 covers the opening the aneurysm A. The rings 14 hold the device 24 in place within the vessel V. Thus, 10 blood flows flowing through the vessel V passes undisturbed through the rings 14 and is blocked from entering the aneurysm A by the covering 18. In some embodiments, the device 24 includes one or more alignment elements, such as a radiopaque filament 26, to determine when the covering 18 is desirably aligned over the aneurysm A. Such alignment may be detected by monitoring the radiopaque filament 26 until it is within the aneurysm A. Other 15 examples of alignment elements include a balloon, which may be inflated into the aneurysm A when desirably aligned, or a fluid port which passes fluid into the aneurysm A when desirably aligned.

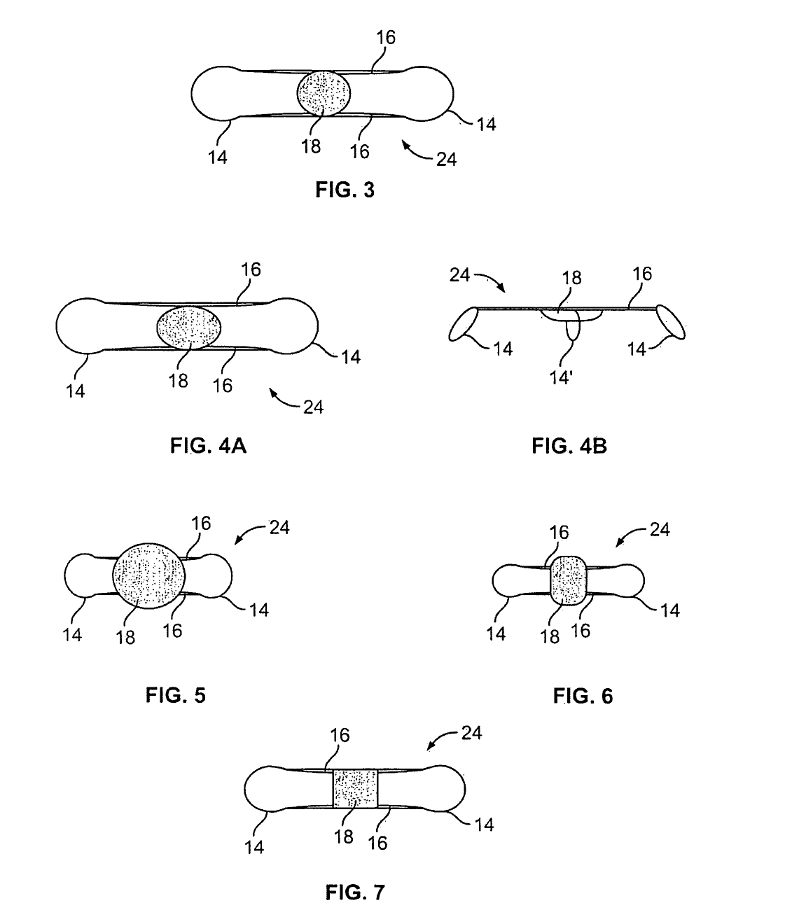

FIG. 3 illustrates another embodiment of a covering device 24 of the present invention. In this embodiment, the number of struts 16 has been minimized to allow for easier delivery. In addition, the rings 14 are C-shaped rather than circular shaped. FIGS. 4A-4B illustrate a similar embodiment. In this embodiment, the device 24 has an oval shaped covering 18, as illustrated in FIG. 4A. FIG. 4B provides a side view of the device 24 of FIG. 4A. In this embodiment, the device 24 includes an additional ring 14′ positioned at or near the covering 18 for added stability near the aneurysm. Such a ring 14′ may optionally be 25 formed from wire or ribbon material. FIGS. 5-7 illustrate alternative embodiments having coverings 18 of different shapes and sizes such as a large oval shape (FIG. 5), a rounded rectangular shape (FIG. 6) and a square shape (FIG. 7). The shape of the covering 18 may be chosen based on a variety of factors, including ease of manufacture or anatomy of the aneurysm, to name a few.

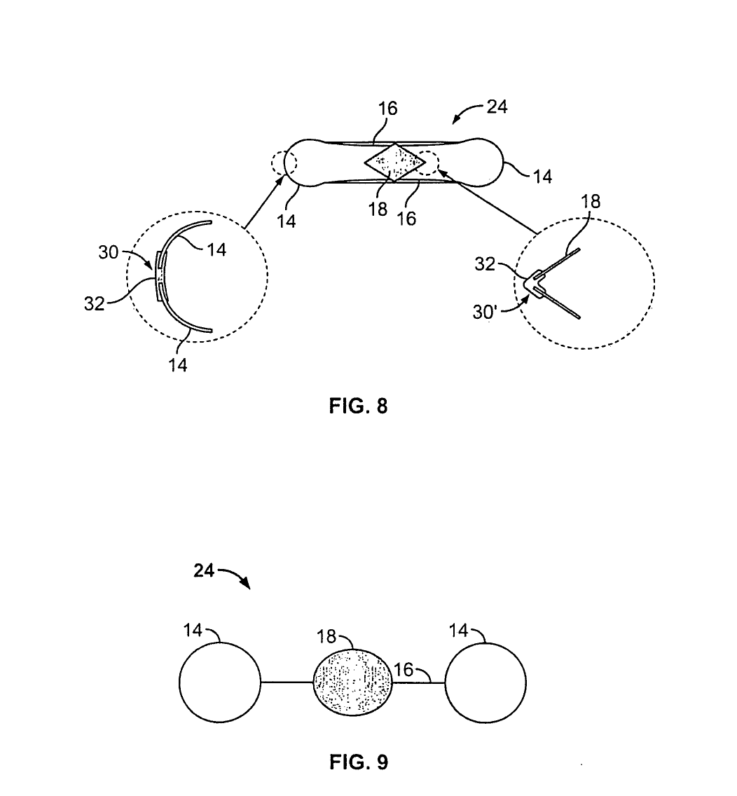

FIG. 8 illustrates another embodiment of the covering device 24. In this embodiment, the covering 18 has a diamond shape. In addition, the device 24 includes a plurality of flexible areas or joints 30, 30′. Joint 30 is shown on a ring 14 and allows flexing or folding of the ring 14. In this embodiment, the joint 30 is formed by joining two discontinuous portions of the ring 14 with a flexible material 32, such as a flexible overtube or elastic. Alternatively, the flexible material 32 may extend along an inside or outside surface of the ring, or a combination of these. A similar joint 30′ is shown on the covering 18. Such joints 30,30′ may improve ease of delivery. Such joints 30,30′ may be used on any of the embodiments of the covering device 24.

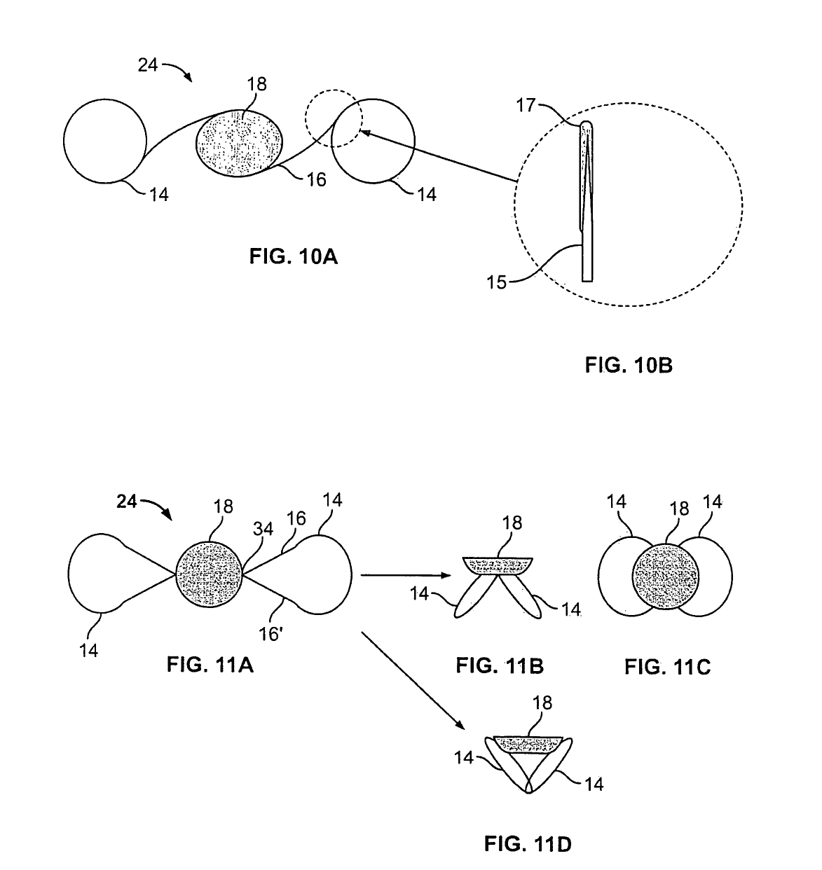

FIG. 9 illustrates another embodiment of a covering device 24. In this embodiment, the device 24 comprises a covering 18 attached to two rings 14, each by a coaxial strut 16. FIG. 10A illustrates a similar embodiment of a covering device 24. Here covering 18 is attached to each ring 14 by a curved strut 16. In addition, the device 24 includes an embodiment of a strain relief mechanism, illustrated in FIG. 10B. As shown, the core 15 tapers and is covered by a softer material 17 so as to maintain the outer diameter. The core 15 may also be one piece with free ends protected by the strain relief mechanism.

FIG. 11A illustrates another embodiment of a covering device 24. Here the device 24 includes two rings 14 having a C-shape, wherein each ring 14 is connected to two struts 16, 16′ which are coupled to the covering 18 at a common location 34. FIG. 11B illustrates one embodiment of the device 24 of FIG. 11A in a deployed position. Here, the struts 16, 16′ (hidden) fold under the covering 18 so that the rings 14 are drawn together under the covering 18. This may allow positioning of the device 24 in blood vessels having minimal space around the aneurysm conducive to anchoring the device 24. FIG. 11C shows a top view of the deployed device 24 wherein the rings 14 only extend a short distance from the covering 18. It may be appreciated that in some embodiments the covering 18 entirely cover the rings 14 when in the deployed position. FIG. 11D shows an alternative deployed position of the device of FIG. 11A. Here, the rings 14 tilt inwards, underneath the covering 18.

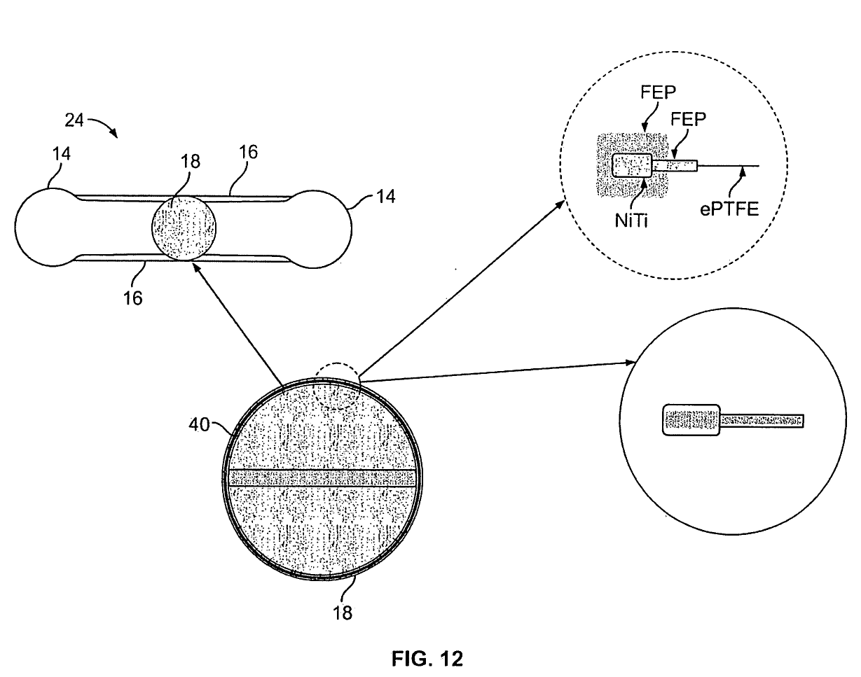

FIG. 12 illustrates some example features which may be included in the covering 18. For example, a slit 40 may be formed by overlapping material. This provides for better folding flexibility and leaves a closed surface once the covering 18 is deployed. If the covering 18 is formed from a scaffold, such as a nitinol hoop, with a flexible covering, such as ePTFE, the scaffold may be coated with a material, such as FEP. The material may be dashed for better folding and flexibility. If the covering 18 is formed from a metal or alloy, the covering '18 may be etched so that the covering 18 is thicker near its edges and thinner near its center.

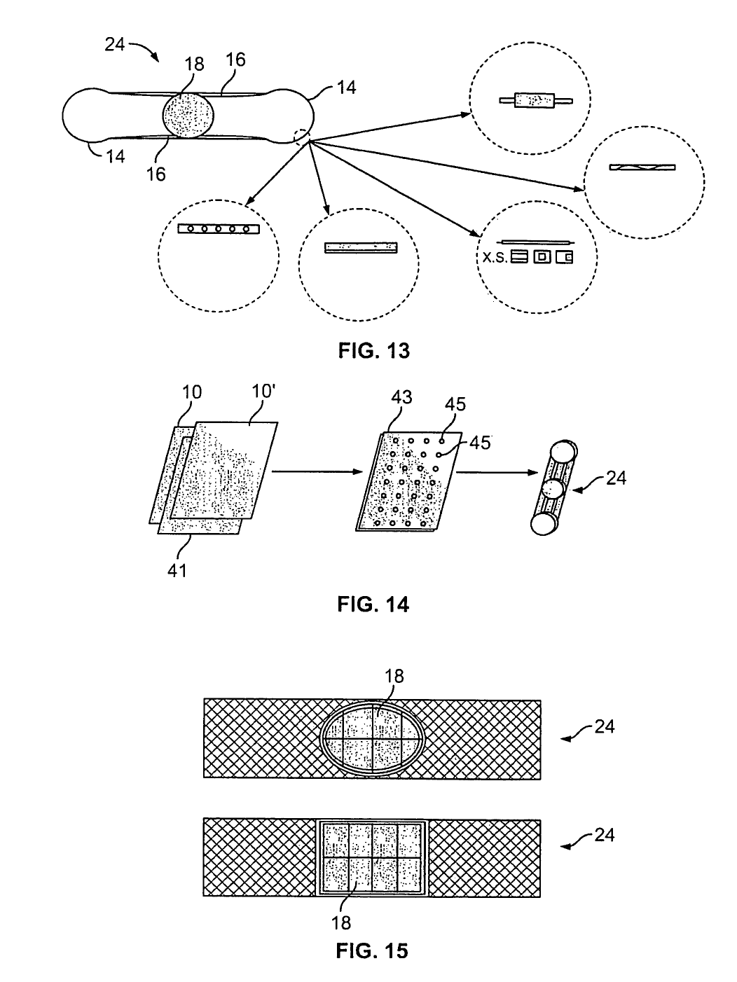

In some embodiments, the covering device 24 provides radiopacity to assist in desired placement of the device 24 within a blood vessel. Any portion of the device 24 may be radiopaque. FIG. 13 illustrates a variety of methods for providing such radiopacity. For example, a radiopaque agent may be deposited in cut channels in the device 24. A radiopaque agent may be chemical, sputtered or ion deposited on the device 24. A radiopaque agent may be incorporated into the device material, woven through the device material, or crimped on the outside of the device material. In some embodiments, the device material is comprised of a “drawn filled tube” (DFT) filled with, for example, gold, or platinum, platinum-iridium.

FIG. 14 illustrates an example of device material having radiopaque material incorporated therein. As shown, a sheet 40 of radiopaque material, such as gold, is sandwiched between two sheets 10, 10′ of material, such as nitinol, stainless steel, tantalum, Co—Cr, or other alloy. The sheets 10, 41, 10′ are joined together to form a laminate 43, such as with welds or pins 45. The covering device 24 is then cutout from the laminate 43 and formed into three dimensions such as described above.

FIG. 15 illustrates an embodiment of a covering device 24 constructed from mesh or a cut tube stent and a covering 18 formed from a flexible material, such as FEP, PTFE, ePTFE, nylon, polyurethane, Tecoflex, Pebax, polyester, PET, Hytrel, to name a few. The covering 18 is adhered to the mesh by using an appropriate weld.

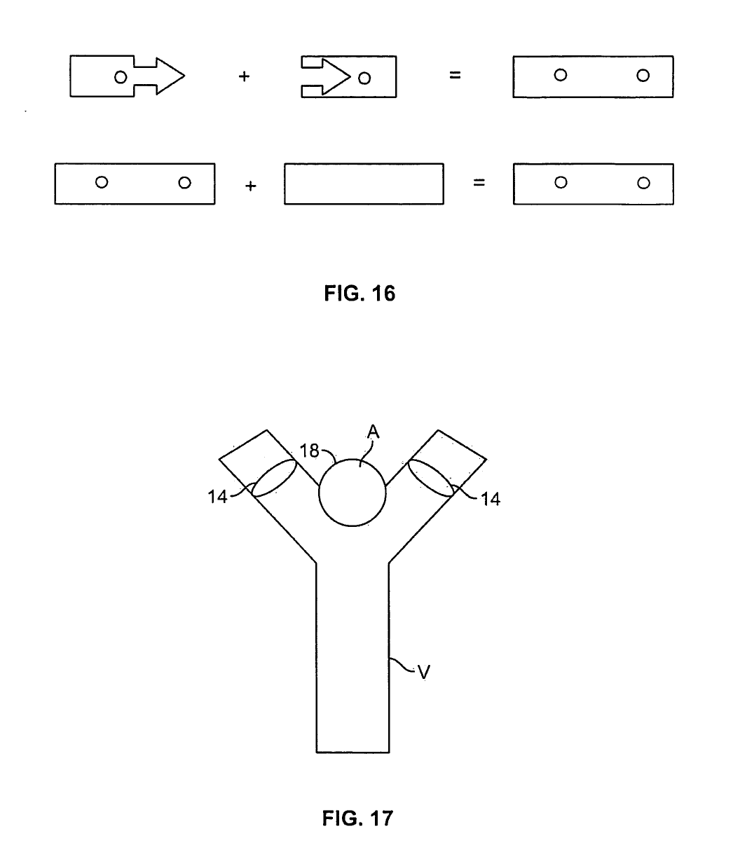

In some embodiments, it may be desired to have some components elastic and some inelastic. It is often the case that these materials cannot be easily connected. FIG. 16 illustrates a method where two such materials can be joined by way of a mechanical fit and then sealed by a pressure fit of a material constraining the surface and keeping the dissimilar pieces locked in position relative to each other. For example, roughening and/or holes may allow for a better hold on each material by increasing friction or flowing into a channel and locking. This is only an example and many others are possible with a similar objective.

It may be appreciated that the covering device 24 may be used to treat aneurysms in a variety of locations. Often, cerebral berry aneurysms are located at bifurcations of blood vessels. The covering devices 24 described herein may be used to treat such aneurysms. FIG. 17 illustrates a covering 24 positioned within a bifurcated blood vessel V so that the covering 18 covers the aneurysm A.

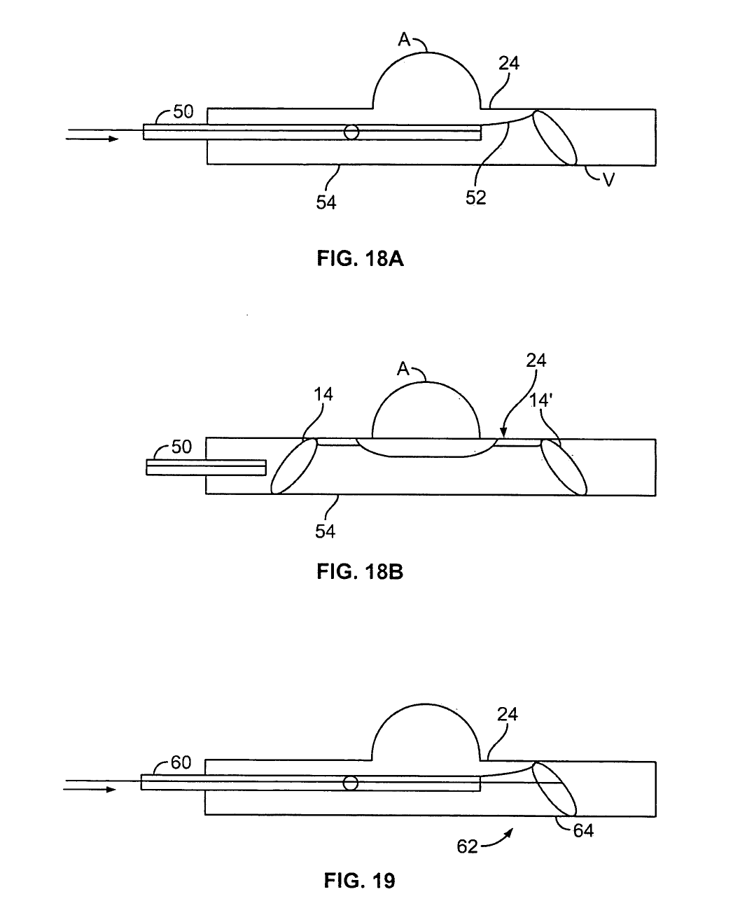

A variety of delivery devices may be used to deliver the covering devices 24 of the present invention. For example, FIGS. 18A-18B illustrate a push-style delivery system. In this embodiment, the delivery system comprises a catheter 50 having a lumen 52 and a push-rod 54 extending through the lumen 52. The covering device 24 is loaded within the lumen 52 near the distal end of the catheter 50. The catheter 50 is then advanced through the vasculature to a target delivery site within a blood vessel V. The covering device 24 is then deployed at the target delivery site by advancing the push-rod 54 which pushes the device 24 out of the lumen 52 and into the blood vessel V. Typically, the device 24 is deployed so that a first ring 14 is disposed on one side of an aneurysm A and a second ring 14′ is disposed on the other side of the aneurysm A, as illustrated in FIG. 18B.

FIG. 19 illustrates a pull-style delivery system. In this embodiment, the delivery system comprises a catheter 60 having a lumen 62 and a pull element 64 extending through the lumen 52. The covering device 24 is loaded within the lumen 62 near the distal end of the catheter 60 and attached to the pull element 64. The catheter 60 is then advanced through the vasculature to a target delivery site within a blood vessel. The covering device 24 is then deployed at the target delivery site by advancing the pull element 64 which pulls the device 24 out of the lumen 52 and into the blood vessel V. Typically, the device 24 is deployed so that a first ring 14 is disposed on one side of an aneurysm A and a second ring 14′ is disposed on the other side of the aneurysm A, such as illustrated in FIG. 18B. It may be appreciated that the pull element 64 may alternatively extend along the exterior of the catheter 60 or through a lumen in the wall of the catheter 60.

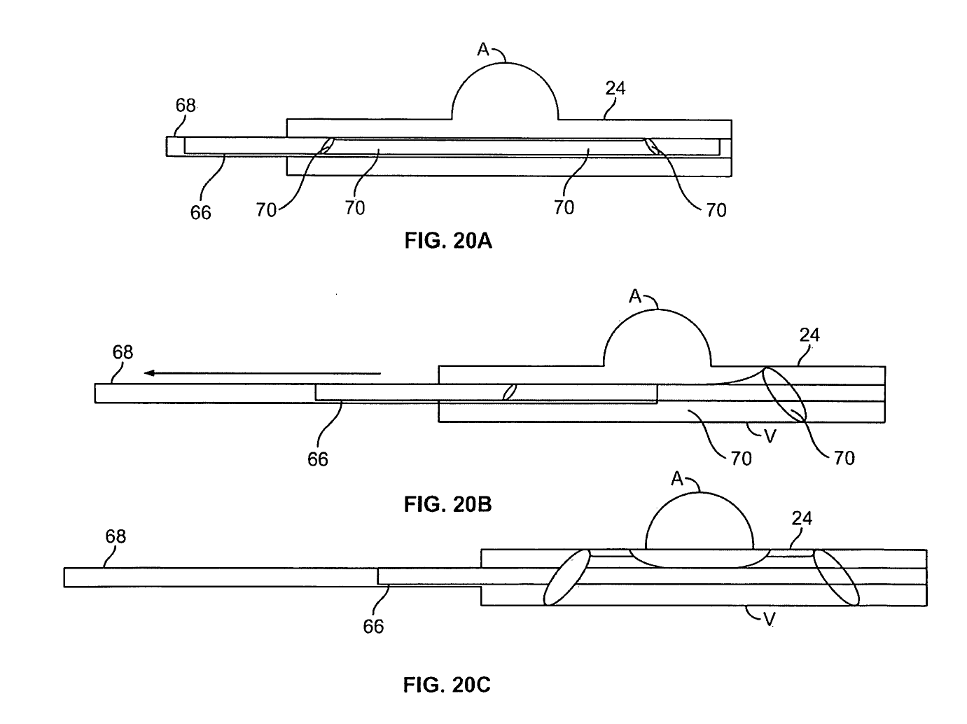

FIGS. 20A-20C illustrate a sheath-style delivery system. In this embodiment, the delivery system comprises a rod 66 positionable within a sheath 68. The covering device 24 is mountable on the rod 66 and the sheath 68 is extendable over the covering device 24, as illustrated in FIG. 20A. The system is then advanced so that the covering 18 of the device 24 is positioned with a blood vessel V so as to cover an aneurysm A. In this embodiment, the rod 66 includes radiopaque markers 70 so assist in such positioning. The sheath 68 is then retracted, as illustrated in FIG. 20B, releasing covering device 24 within the blood vessel V. Once the device 24 is deployed, as illustrated in FIG. 20C, the rod 66 may then be retracted leaving the device 24 in place.

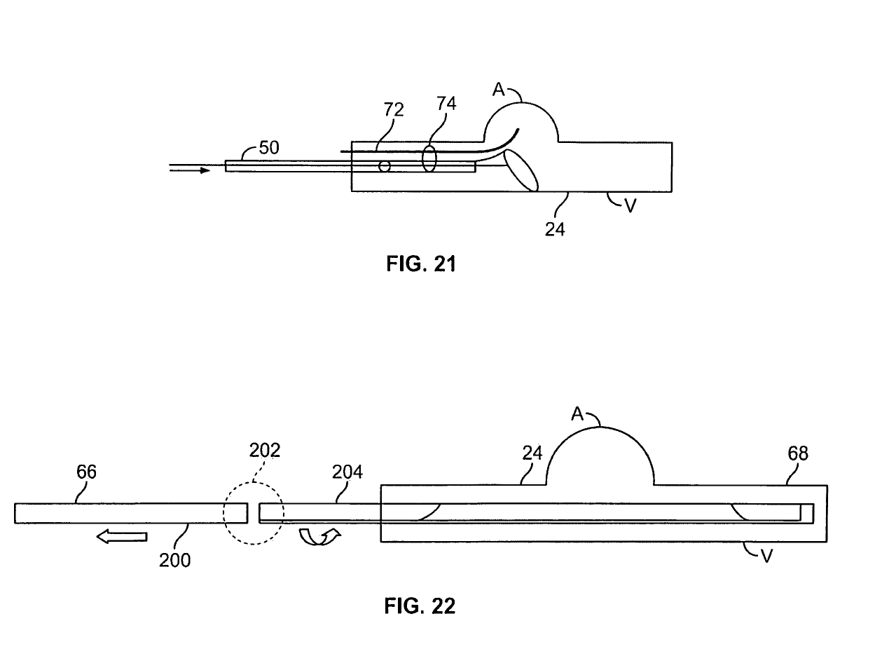

In some embodiments, the delivery system may be guided to the desired target location within the blood vessel with the use of a guidewire. FIG. 21 illustrates the positioning of a guidewire 72 within an aneurysm A in a blood vessel V. The delivery system, such as the push-style delivery system described above, includes a guide 74 that is used to track over the guidewire 72 to the target location. In this example, the guide 74 comprises a loop that is mounted on the catheter 50. Once the catheter 50 is positioned at the target location, the device 24 is deployed as described above. It may be appreciated that a guide 74 may be used with any delivery system.

In some embodiments, the delivery system provides for adjustment of the orientation of the covering device 24 to assist in desired covering of the aneurysm A. In particular, the delivery system provides for rotation of the covering device 24 within the blood vessel V so as to desirably align the covering 18 with the aneurysm A. Typically, such rotation is challenging because the vascular anatomy preceding most intracranial aneurysms is tortuous and long and therefore difficult to control and transmit torque for precise delivery. However, the present invention provides a variety of mechanisms for transforming longitudinal forces to rotational forces. This is desirable since longitudinal forces are often easier to transmit over long distances and through curves, or may be more precise than rotational forces. Example longitudinal forces include pull or push forces from an actuator wire, fiber, braid or other element to move, for example, piston gears to create a rotational step. Alternatively, pressure from hydraulic or gas forces may be converted to move a piston. Actual rotation occurs over a much shorter distance and may be ratcheted for easier control. It may be appreciated that the delivery systems described herein may be used to deliver a variety of devices and are not limited to the delivery of covering devices. In particular, the delivery systems which provide for rotation of the covering device may be used to deliver any device which may benefit from rotation or adjustment of orientation.

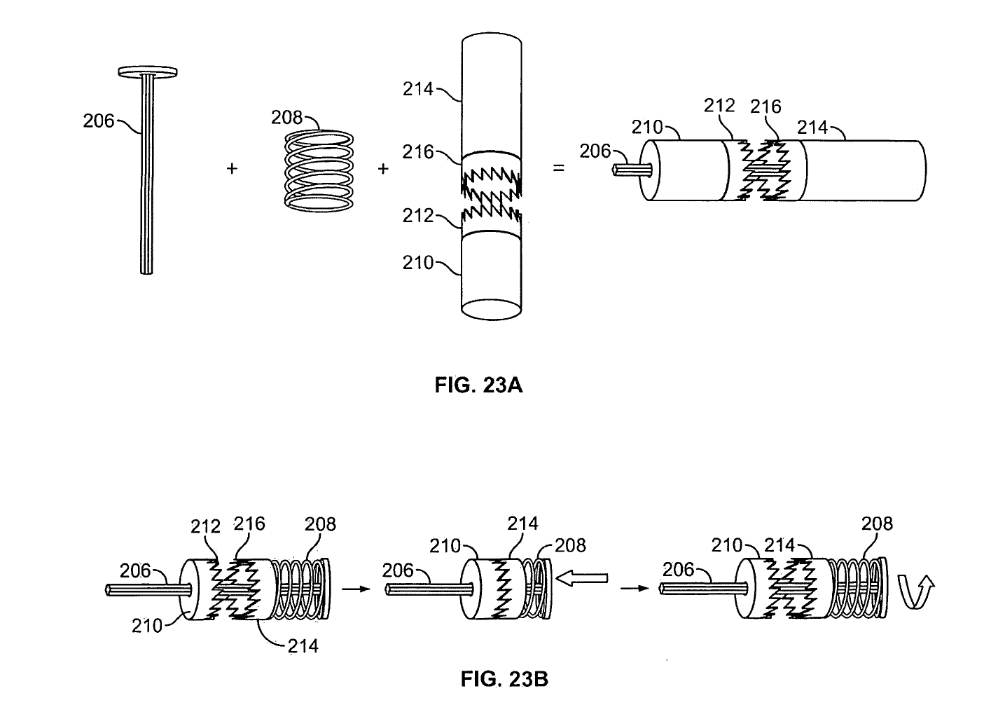

An example of such a delivery system is illustrated in FIG. 22 and FIGS. 23A-23B. FIG. 22 illustrates a sheath-style delivery system similar to FIGS. 20A-20C. Here, the delivery system comprises a rod 66 positionable within a sheath 68. The rod 66 includes a proximal end 200, a force conversion mechanism 202 and a coupling device 204. The covering device 24 is mountable on the coupling device 204 and the sheath 68 is extendable over the rod 66 and covering device 24. The system is then advanced so that the covering 18 of the device 24 is positioned within a blood vessel V. The force conversion mechanism 202 converts longitudinal force applied to the proximal end 200 of the rod 66 to rotational force applied to the coupling device 204 so as to rotate the deliverable device. FIGS. 23A-23B illustrate an embodiment the force conversion mechanism 202. Here, the mechanism 202 comprises a piston 206, a spring 208, a proximal rotor 210 having teeth 212 and a distal rotor 214 having angled teeth 216. The proximal rotor 210 and distal rotor 214 are held apart by the spring 208. Applying longitudinal force to the piston 206, compresses the spring 208 drawing the rotors 210, 212 together. The teeth 212 of the proximal rotor 210 engage the angled teeth 216 of the distal rotor 214 causing the distal rotor 212 to rotate. Each time the piston 206 and proximal rotor 210 are translated to engage the distal rotor 212, the distal rotor 212 turns one tooth dimension. The amount of distal rotation, therefore, can be selectively controlled by design of the gear teeth and the number of actuations engaged. In this embodiment, the distal rotor 214 is joined with or forms the coupling device 204 so as to rotate the covering 18 to cover an aneurysm A. The sheath 68 is then retracted, releasing covering device 24 within the blood vessel V.

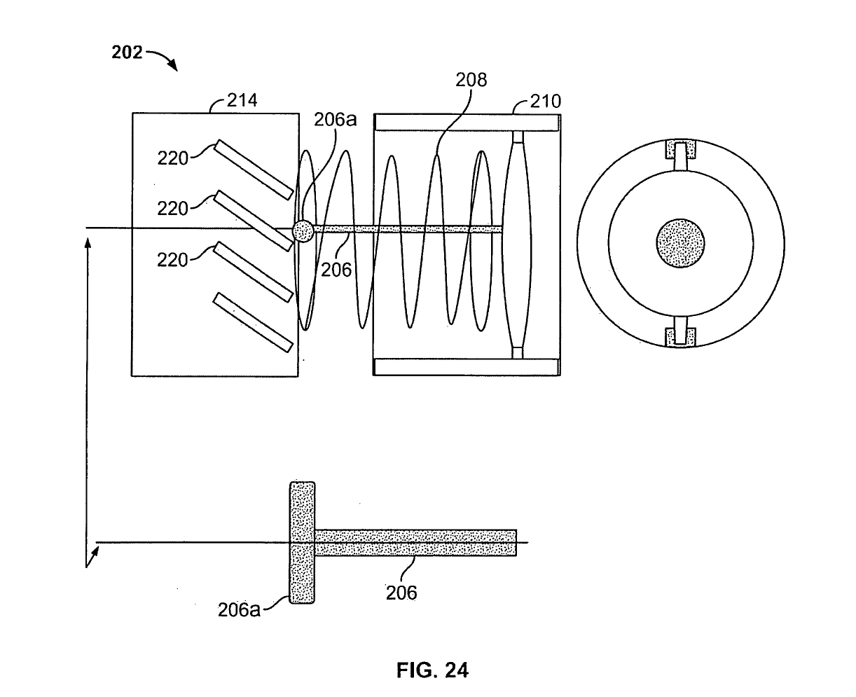

FIG. 24 illustrates another embodiment of a force conversion mechanism 202. Here, the mechanism 202 comprises a piston 206 having a barbell end 206a, a spring 208, a proximal rotor 210 and a distal rotor 214. The distal rotor 214 has internal ridges 220 having a curved or slanted orientation around the inner circumference. The piston 206 passes through the rotors 210,214 and spring 208 so that the barbell end 206a is disposed between a pair of ridges 220. Longitudinal force applied to the piston 206 pushes the barbell end 206a along the ridges 220. The slant of the ridges 220, rotates the distal rotor 214. The barbell end 206 eventually extends beyond the ridges 220. Once the piston 206 is released, the spring 208 draws the piston 206 back between an adjacent set of ridges 220 in the new rotated position. This can be repeated to further rotate the distal rotor 214.

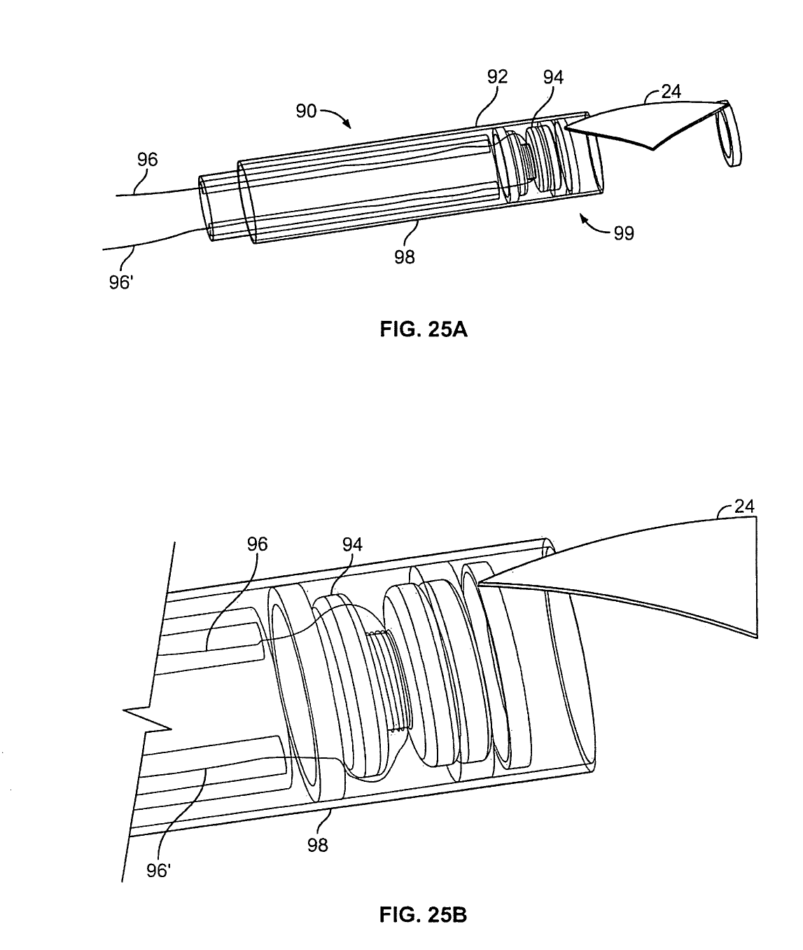

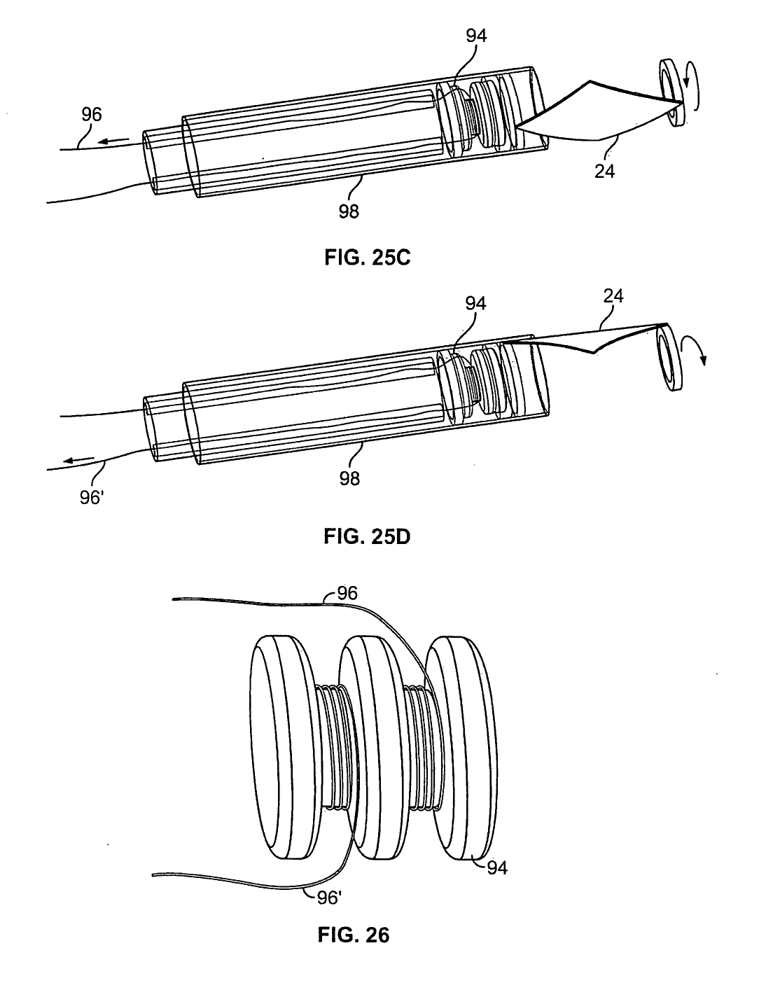

FIGS. 25A-25D illustrate an embodiment of a delivery system 90 which provides for rotation of the covering device 24. In this embodiment, as shown in FIG. 25A, the delivery system 90 includes a force conversion mechanism 92 which is attached or coupled with a coupling device which is removably coupleable with the covering device 24. The force conversion mechanism 92 comprises a pulley 94 which is rotated with the use of two tension wires 96, 96′. Referring to FIG. 25B, one tension wire 96 is wrapped around the pulley 94 in a clockwise direction and the other tension wire 96′ is wrapped around the pulley 94 in a counterclockwise direction. Typically the force conversion mechanism 92 is disposed within a catheter 98 or similar elongate delivery device so as to be removably coupled with the covering device 24 which is deployable therefrom. Therefore, at least a portion of the force conversion mechanism 92 is disposed near a distal end 99 of the catheter 98. Each of the wires 96, 96′ extend along the length of the catheter 98, optionally within individual lumens. Pulling or applying longitudinal force to one tension wire 96 causes the pulley 94 and therefore the covering device 24 to rotate in a counterclockwise direction, as shown in FIG. 25C. And, pulling or applying longitudinal force to the other tension wire 96′ causes pulley 94 and therefore the covering device 24 to rotate in a clockwise direction, as shown in FIG. 25D. The wires 96,96′ may be wrapped around a common portion of the pulley 94, as illustrated in FIGS. 25A-25D, or each wire may be wrapped around a separate portion of the pulley, as illustrated in FIG. 26.

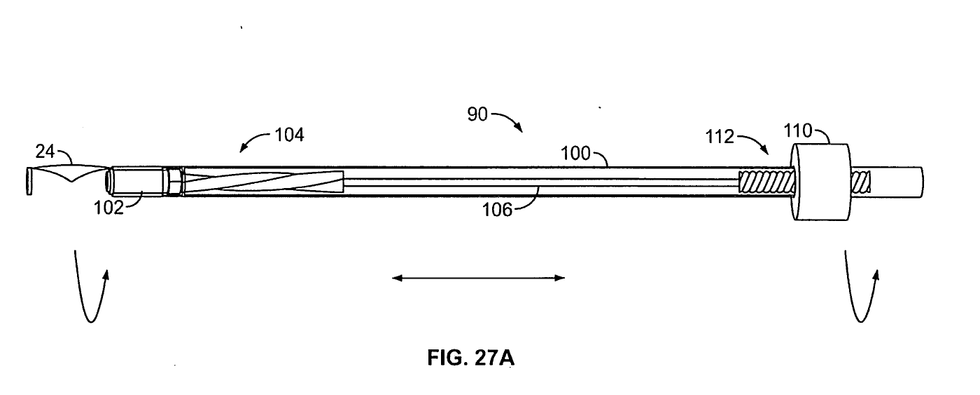

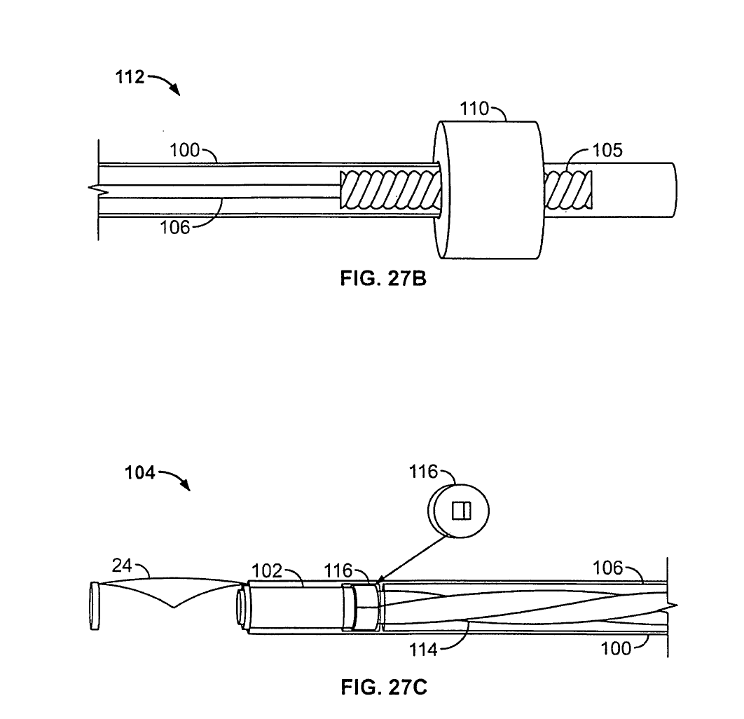

FIGS. 27A-27C illustrate another embodiment of a delivery system 90 which provides for rotation of the covering device 24. In this embodiment, as shown in FIG. 27A, the delivery system 90 comprises a catheter 100 or similar elongate delivery device having a coupling member 102 near its distal end 104 which is configured to be removably coupled with the covering device 24. The coupling member 102 is connected with a force conversion mechanism comprising an elongate shaft 106 which is longitudinally translatable within the catheter 100. Such translation is achieved by rotating a knob 110 near the proximal end 112 of the catheter 100. FIG. 27B illustrates the proximal end 112 of the catheter 100. As shown, the knob 110 has a threaded interior which mates with a threaded portion 105 of the shaft 106. Thus, rotation of the knob 110 causes translation of the shaft 106. FIG. 27C illustrates the distal end 104 of the catheter 100. The coupling member 102 is shown coupled with the covering device 24. The coupling member 102 is also connected with a twisted shaped portion 114 of the shaft 106 via a shaped register 116. In this embodiment, the twisted shaped portion 114 comprises a twisted square portion and the shaped register 116 comprises a square register. However, any shape, such a triangle, rectangle, star, etc., which mates with purchase may be used. As the shaft 106 is translated in a distal direction, the twisted shaped portion 114 is advanced through the shaped register 116. The twisted configuration of the twisted shaped portion 114 rotates the square register 116 which in turn rotates the coupling member 102 and covering device 24. The covering device 24 may be rotated in the opposite direction by rotation the knob 110 in the opposite direction.

The rotational aspects of the delivery systems of the present invention assist in positioning the covering 18 of the covering device 24 over the neck of the aneurysm so as to block flow into the aneurysm. Additional features may also be used to assist in desired positioning of the covering 18. For example, FIGS. 2C-2D illustrate a delivery system 120 having a fill tube 122 which couples with the covering device 24. In this embodiment, the fill tube 120 couples with a port 124 through the covering 18 so that fluid 126 flowing through the fill tube 120 passes through the port 124. Referring to FIG. 2C, when the covering 18 is aligned with an aneurysm A so that the aneurysm A is desirably isolated from the blood vessel V, fluid 126 flowing through the fill tube 120 will fill the aneurysm A. By using a radiopaque fluid 126, such filling of the aneurysm A may be visualized, thus verifying that the covering device 24 is desirably placed. If the covering device 24 is improperly placed, such that the aneurysm A is not sufficiently isolated, the fluid 126 will extravisate from the aneurysm A, as illustrated in FIG. 2D. The covering device 24 may then be repositioned until desirably placed.

Although the foregoing invention has been described in some detail by way of illustration and example, for purposes of clarity of understanding, it will be obvious that various alternatives, modifications and equivalents may be used and the above description should not be taken as limiting in scope of the invention which is defined by the appended.

What is claimed is:

1. A covering device for isolating an aneurysm within a blood vessel comprising:
a covering; and a structure having a three dimensional shape for supporting the covering within the blood vessel so as to isolate the aneurysm, wherein the structure has been cut from a sheet and formed into its three dimensional shape.

2. A device as in claim 1, wherein the structure comprises at least one ring and at least one strut which are disposed in the same plane within the sheet and disposed in different planes when formed into the three dimensional shape.

3. A device as in claim 2, wherein the at least one ring is arranged substantially concentric with the blood vessel when supporting the covering within the blood vessel.

4. A device as in claim 1, wherein the covering and the structure are formed from a single cutout from the sheet.

5. A delivery system for adjusting the orientation of a deliverable device within a blood vessel comprising:
an elongate catheter having a proximal end and a distal end;
a coupling device disposed near the distal end of the catheter configured to removably couple with the deliverable device; and
a force conversion mechanism coupled with the coupling device which converts a longitudinal force applied near the proximal end of the catheter to a rotation force applied to the coupling device so as to rotate the deliverable device.

6. A delivery system as in claim 5, wherein the force conversion mechanism comprises a pulley and at least one tension wire extending from the pulley toward the proximal end of the catheter.

7. A delivery system as in claim 5, wherein the force conversion mechanism comprises an elongate shaft which is longitudinally translatable within the catheter and has a twisted shaped portion which rotates the coupling device as the shaft longitudinally translates.

8. A delivery system as in claim 5, wherein the force conversion mechanism comprises a pair of rotors, wherein the longitudinal force engages the rotors and such engagement rotates the coupling device.
